(19) United States
(12) Patent Application Publication
(10) Pub. No.: US 2022/0273929 A1
(43) Pub. Date: Sep. 1, 2022

(54) SYSTEMS AND METHODS FOR OPENING TISSUES

(71) Applicant: THE TRUSTEES OF COLUMBIA UNIVERSITY IN THE CITY OF NEW YORK, New York, NY (US)

(72) Inventor: Elisa E. Konofagou, New York, NY (US)

(73) Assignee: THE TRUSTEES OF COLUMBIA UNIVERSITY IN THE CITY OF NEW YORK, New York, NY (US)

(21) Appl. No.: 17/726,193

(22) Filed: Apr. 21, 2022

Related U.S. Application Data
(63) Continuation of application No. PCT/US2020/057130, filed on Oct. 23, 2020.
(60) Provisional application No. 62/925,094, filed on Oct. 23, 2019.

(51) Int. Cl. A61M 37/00; A61B 8/08

(52) U.S. Cl. CPC A61M 37/0092; A61B 8/481; A61N 2007/0039

(57) ABSTRACT

The present subject matter relates to techniques for opening target tissue. The disclosed system can include a navigation guidance device configured to locate and/or monitor the target tissue, a single-element transducer for stimulating the target tissue with focused ultrasound (FUS), and a processor configured to determine a cavitation mode. The navigation guidance device can include a cavitation detector and an arm. The single-element transducer can be attached to the arm and be configured to induce the FUS with a predetermined parameter to open the target tissue.

### CROSS-REFERENCE TO RELATED APPLICATION

This application is a continuation of International Patent Application No. PCT/US2020/057130 filed Oct. 23, 2020, which claims priority to U.S. Provisional Application No. 62/925,094, which was filed on Oct. 23, 2019, the entire contents of which are incorporated by reference herein.

### GRANT INFORMATION

This invention was made with government support under grant numbers R01-EB009041 and R01-AG038961 awarded by the National Institutes of Health. The government has certain rights in the invention.

### BACKGROUND

Focused ultrasound (FUS) can be a non-invasive and non-ionizing therapeutic technique for lithotripsy, tumor ablation, neuromodulation, and essential tremor treatment. Microbubbles can be used as contrast agents in ultrasound imaging and as stress mediators in ultrasound therapy to deliver drugs into cells, tumors, or tissues.

Certain FUS techniques can be performed for non-invasive and reversible blood-brain barrier (BBB) opening. The FUS-mediated BBB opening can be performed in animal models, from rodents non-human primates (NHPs). Certain clinical trials have been performed on a human subject, e.g., by fixing devices within the skull bone and connected to an external power supply via a transdermal needle. Certain techniques involve the generation of FUS through a hemispherical array embedded within the MRI bore. Such multi-element arrays can be configured to simultaneous treatment monitoring and planning based on computed tomography (CT) scans of the treated subject. However, these techniques can be complex and require additional medical devices (e.g., CT and MRI) for inducing the FUS and monitoring. Furthermore, certain FUS techniques can induce certain types of damage to tissues and fail to provide safe long-term treatments.

Therefore, there is a need for simple FUS techniques that can be used for opening tissues with improved safety and efficiency. +

### SUMMARY

The disclosed subject matter provides techniques for opening target tissue. The disclosed subject matter provides systems and methods for opening target tissue with focused ultrasound (FUS).

In certain embodiments, the disclosed system can include a navigation guidance device, a single element transducer, and a processor. In non-limiting embodiments, the navigation guidance device can be configured to locate and/or monitor the target tissue. In some embodiments, the single element can be configured to induce FUS with a predetermined parameter to open the target tissue. In non-limiting embodiments, the processor can be configured to determine a cavitation mode.

In certain embodiments, the navigation guidance can include a cavitation detector and an arm. The cavitation detector can be configured to capture a cavitation signal. The cavitation signal can be a cavitation magnitude, a cavitation duration, and/or a microbubble velocity. In non-limiting embodiments, the cavitation detector can be configured to detect the microbubble cavitation. In some embodiments, the arm can be configured to have 4 degrees of freedom and be controlled by a controller. In non-limiting embodiments, the navigation guidance device can be an image-based navigator device.

In certain embodiments, the single element transducer can be connected to a function generator to induce FUS with the predetermined parameter. The predetermined parameter to open the target tissue can be selected from the group consisting of a center frequency, an outer diameter, an inner diameter, a radius of curvature, and a combination thereof. In non-limiting embodiments, the center frequency can range from about 0.2 MHz to about 0.35 MHz. In some embodiments, the outer diameter ranges from about 60 mm to about 110 mm. In non-limiting embodiments, the radius of curvature can range from about 70 mm to about 110 mm. The inner diameter can be about 44 mm. In certain embodiments, the single element transducer can be connected to the arm of the navigation guidance device.

In certain embodiments, the processor can be configured to determine a cavitation mode. The processor can be configured to determine a stable cavitation dose (SCD) and an inertial cavitation dose (ICD) based on the cavitation signal. In non-limiting embodiments, the processor can be configured to determine a value of the predetermined parameter through numerical simulations.

In certain embodiments, the target tissue can include a cortical brain structure, a subcortical brain structure, or a combination thereof.

In certain embodiments, the disclosed subject matter provides a method for opening target tissue. The method can include locating the target tissue using a navigation guidance device, administering microbubbles into the target tissue, and applying FUS using a single element transducer. In non-limiting embodiments, the navigation guidance device comprises a cavitation detector and an arm. In some embodiments, the single element transducer can induce the FUS with a predetermined parameter to open the target tissue. The predetermined parameter can be a center frequency, an outer diameter, an inner diameter, a radius of curvature, and a combination thereof.

In certain embodiments, the method can further include obtaining a cavitation signal using the cavitation detector. The cavitation signal can be a cavitation magnitude, a cavitation duration, and/or a microbubble velocity.

In certain embodiments, the method can further include determining a cavitation mode by calculating a stable cavitation dose (SCD) and an inertial cavitation dose (ICD) based on the cavitation signal.

In certain embodiments, the method can further include determining the predetermined parameter by performing numerical simulations.

The disclosed subject matter will be further described below.

### BRIEF DESCRIPTION OF THE DRAWINGS

FIG. 1 provides a photograph of an example system in accordance with the disclosed subject matter.

FIG. 2 provides images showing an example numerical simulation of ultrasound propagation with different single element-transducers (top to bottom) in accordance with the disclosed subject matter.

FIG. 3 provides images showing an example numerical simulation of ultrasound propagation with the focused ultrasound transducer targeting structures of variable depth within a human skull in accordance with the disclosed subject matter.

FIG. 4 provides graphs showing lateral (top) and axial (bottom) profiles of the simulated pressure field within a human skill in accordance with the disclosed subject matter.

FIGS. 5A-5C provide graphs showing simulated human skull-induced focal distortion. FIG. 5A shows a graph showing a full width at half maximum (FHWM) change caused by the presence of the human skull. FIG. 5B provides a graph showing simulated focal shifts along the axial and lateral dimensions. FIG. 5C provides a graph showing average focal shifts across the lateral and axial dimensions.

FIGS. 6A-6E provide diagrams and graphs showing human skull-induced focal distortion. FIG. 6A provides a diagram showing an example system for measuring focal distortion using a hydrophone. A raster scan can be performed to measure the focal volume in (FIGS. 6B and 6C—left side) free field and (FIGS. 6B and 6C—right side) with a human skull fragment. FIG. 6D provides a graph showing a full width at half maximum change. FIG. 6E provides a graph showing focal shifts along the lateral and axial dimensions.

FIGS. 7A-7D provide diagrams and graphs showing passive cavitation detection through the human skull. FIG. 7A provides a diagram showing an example In vitro system for passive cavitation detection. FIG. 7B provides graphs showing spectra of control and microbubble acoustic emissions for mechanical indexes (MIs) of 0.4 (left), 0.6 (middle), and 0.8 (right) in free-field. FIG. 7C provides graphs showing spectra of control and microbubble acoustic emissions through the human skull. FIG. 7D provides graphs showing cavitation levels in free-field and through the human skull for control and microbubbles, at MIs of 0.4 (left), 0.6 (middle), and 0.8 (right).

FIG. 8 provides a graph showing skull heating using a focused ultrasound transducer at mechanical indexes (MIs) of 0.4, 0.6, and 0.8 and clinically relevant ultrasound parameters (center frequency: 0.25 MHz, pulse length: 2500 cycles or 10 ms, pulse repetition frequency: 2 Hz, duty cycle: 2%, total duration: 2 min).

FIG. 9 provides images showing the opening of the blood-brain barrier (BBB) in a non-human primate (NHP) model.

FIGS. 10A-10I provide graphs showing In vivo passive cavitation detection measurements. FIG. 10A shows a spectral amplitude of non-human primate (NHP) 1 before microbubble injection. FIG. 10B shows a spectral amplitude of NHP 1 after microbubble injection. FIG. 10C shows a spectrogram of the entire treatment session for NHP 1. FIG. 10D shows a spectral amplitude of non-human primate (NHP) 2 before microbubble injection. FIG. 10E shows a spectral amplitude of NHP 2 after microbubble injection. FIG. 10F shows a spectrogram of the entire treatment session for NHP 2. FIG. 10G shows stable harmonic cavitation levels of NHP 1 (g). FIG. 10H shows stable harmonic cavitation levels of NHP 2. FIG. 10I shows an average stable harmonic, a stable ultraharmonic, and an inertial cavitation dose during focused ultrasound treatment for NHP 1 (filled bars) and NHP 2 (patterned bars), following microbubble administration (t>15 s).

It is to be understood that both the foregoing general description and the following detailed description are exemplary and are intended to provide further explanation of the disclosed subject matter.

### DETAILED DESCRIPTION

The disclosed subject matter provides techniques for opening target tissue. The disclosed subject matter provides systems and methods for opening target tissue using focused ultrasound (FUS). The disclosed subject matter provides certain FUS parameters, which can allow improved attenuation and distortion of the ultrasound beam, and be suitable for humans.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like refer to obtaining a desired pharmacologic and/or physiologic effect. The effect can be prophylactic in terms of completely or partially preventing a disease or condition or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

In certain embodiments, the disclosed subject matter provides a system for opening target tissue. An example system 100 can include a navigation guidance device and a single element transducer, and a processor. In non-limiting embodiments, the navigation guidance device can include a cavitation detector and an arm.

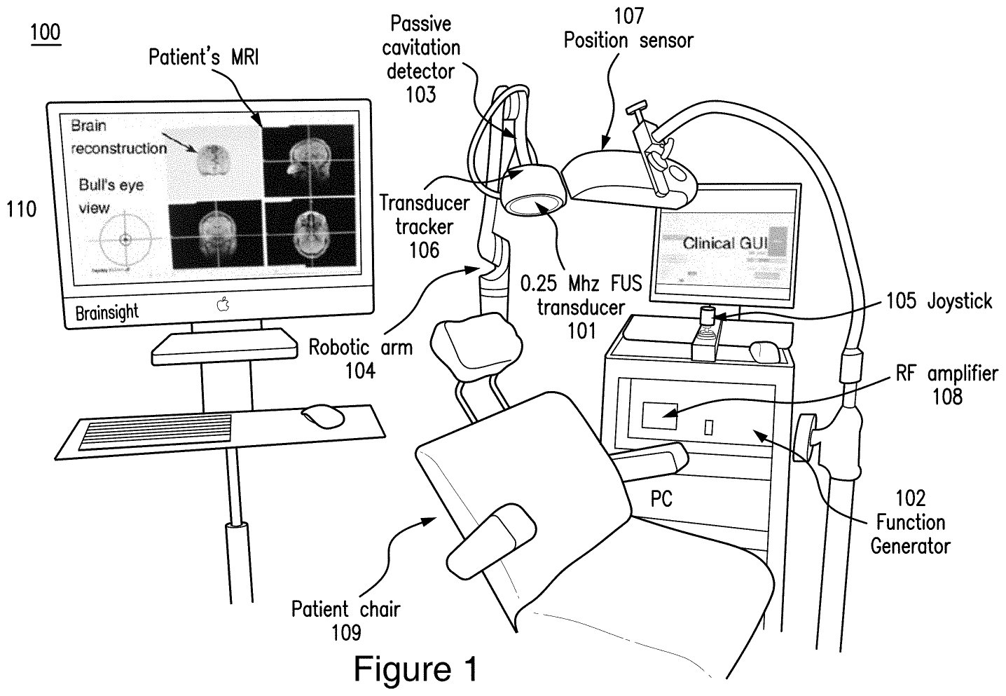

In certain embodiments, the single element transducer 101 can be configured to induce FUS for opening target tissue (FIG. 1). For example, the single element transducer can generate an acoustic radiation force and induce cavitation at the target tissue. The single-element transducer can be connected to a function generator 102 and have a predetermined ultrasound parameter to induce cavitation and open the target tissue. In non-limiting embodiments, the parameters can be modified or adjusted depending on the target tissue or subject.

In certain embodiments, the predetermined ultrasound parameter can include a center frequency. The center frequency can range from about 20 kilohertz (kHz) to about 1 megahertz (MHz). In non-limiting embodiments, the center frequency can range from about 0.1 MHz to about 1 MHz, from about 0.1 MHz to about 0.5 MHz, from about 0.1 MHz to about 0.35 MHz, from about 0.2 MHz to about 0.35 MHz, or from about 0.2 MHz to about 0.25 MHz. In non-limiting embodiments, the center frequency of the FUS stimulation probe can be about 0.2, 0.25, or 0.35 MHz. The disclosed subject matter can improve certain aberration and attenuation caused by the human skull at certain frequency ranges.

In certain embodiments, the predetermined ultrasound parameter can include outer diameter, inner diameter, and radius curvature of the disclosed single element transducer. The outer diameter of the single element transducer can range from about 30 millimeters (mm) to about 200 mm, from about 30 mm to about 150 mm, from about 30 mm to about 110 mm, from about 40 mm to about 110 mm, from about 50 mm to about 110 mm, or from about 60 mm to about 110 mm. In non-limiting embodiments, the outer diameter of the single element transducer can be about 60 or 110 mm. In some embodiments, the inner diameter of the single element transducer can range from about 10 (mm) to about 60 mm, from about 10 mm to about 50 mm, from about 20 mm to about 50 mm, or from about 30 mm to about 50. In non-limiting embodiments, the inner diameter of the single element transducer can be about 44 mm. In some embodiments, the radius of curvature can range from about 30 millimeters (mm) to about 200 mm, from about 30 mm to about 150 mm, from about 30 mm to about 110 mm, from about 40 mm to about 110 mm, from about 50 mm to about 110 mm, from about 60 mm to about 110 mm, or from about 70 mm to about 110 mm. In non-limiting embodiments, the radius curvature can be about 70, 76, or 110 mm.

In certain embodiments, the predetermined ultrasound parameter can include a mechanical index, pulse length, pulse repetition frequency, peak-negative pressure, and sonication duration. The mechanical index can range from about 0.1 to about 1.9, from about 0.1 to about 1.5, from about 0.1 to about 1.0, from about 0.1 to about 0.9, from about 0.1 to about 0.8, from about 0.1 to about 0.7, from about 0.2 to about 0.7, from about 0.3 to about 0.7, or from about 0.4 to about 0.7. In non-limiting embodiments, the mechanical index can be about 0.4 or 0.8. The pulse length can range from about 0.001 milliseconds (ms) to about 100 ms, from about 0.001 ms to about 90 ms, from 0.001 ms to about 80 ms, from 0.001 ms to about 70 ms, from 0.001 ms to about 60 ms, from 0.001 ms to about 50 ms, from 0.001 ms to about 40 ms, from 0.001 ms to about 30 ms, from 0.001 ms to about 20 ms, or from 0.001 ms to about 10 ms. In non-limiting embodiments, the pulse length can be about 10 ms. The pulse length can also range from about 1 cycle to about 5000 cycles, from about 1 cycle to about 4000 cycles, from about 1 cycle to about 10,000 cycles, from about 1 cycle to about 5000 cycles, from about 1 cycle to about 4000 cycles, from about 1 cycle to about 3000 cycles, from about 1 cycle to about 2500 cycles, from about 500 cycles to about 2500 cycles, from about 1000 cycles to about 2500 cycles, from about 1500 cycles to about 2500 cycles, or from about 2000 cycles to about 2500 cycles. In non-limiting embodiments, the pulse length can be about 2500 cycles. The pulse repetition frequency can range from about 0.1 Hz to about 10 kHz, from about 0.1 Hz to about 9 kHz, from about 0.1 Hz to about 8 kHz, from about 0.1 Hz to about 7 kHz, from about 0.1 Hz to about 6 kHz, from about 0.1 Hz to about 5 kHz, from about 0.1 Hz to about 4 kHz, from about 0.1 Hz to about 3 kHz, or from about 0.1 Hz to about 2 kHz. In non-limiting embodiments, the pulse repetition frequency can be about 2 Hz.

In certain embodiments, the sonication duration can range from about 0.1 minutes to about 5 minutes, from about 0.1 minutes to about 4 minutes, from about 0.1 minutes to about 3 minutes, from about 0.1 minutes to about 2 minutes, from about 0.5 minutes to about 2 minutes, or from about 1 minute to about 2 minutes. In non-limiting embodiments, the sonication duration can be about 2 minutes.

In certain embodiments, the peak-negative pressure can range from about 0.1 MPa to about 10 MPa, from about 0.1 MPa to about 9 MPa, from about 0.1 MPa to about 8 MPa, from about 0.1 MPa to about 7 MPa, from about 0.1 MPa to about 6 MPa, from about 0.1 MPa to about 5 MPa, from about 0.1 MPa to about 4 MPa, from about 0.1 MPa to about 3 MPa, from about 0.1 MPa to about 2 MPa, from about 0.1 MPa to about 1 MPa, from about 0.1 MPa to about 0.5 MPa, from about 0.1 MPa to about 0.4 MPa, from about 0.1 MPa to about 0.3 MPa, or from about 0.1 MPa to about 0.2 MPa. In non-limiting embodiments, the peak-negative pressure can be about 0.2 MPa.

In certain embodiments, certain parameters (e.g., acoustic intensity, mechanical index, peak negative pressure) can be derated using subject-specific numerical simulations. Derated pressure refers to the pressure after propagation through the human skull. The attenuation factor can be estimated through numerical simulations.

In certain embodiments, the disclosed system can include microbubbles. The microbubbles can be configured to react to a predetermined pulse of the FUS and induce cavitation for opening the target tissue. The size of the microbubbles can range from about 1 micron to about 10 microns, from about 1 micron to about 9 microns, from about 1 micron to about 8 microns, from about 1 micron to about 7 microns, from about 1 micron to about 6 microns, from about 1 micron to about 6 microns, from about 1 micron to about 5 microns, from about 2 microns to about 5 microns, from about 3 microns to about 5 microns, or from about 4 microns to about 5 microns. In non-limiting embodiments, the size of the microbubbles can be about 1.2, about 4, or about 5 microns. In some embodiments, the dose of the microbubbles can be adjusted depending on a subject. For example, clinical does (e.g., about 10 μl/kg) of the microbubbles for ultrasound imaging applications can be administered into a human subject.

In certain embodiments, the microbubbles are configured to carry or be coated with an active agent. The microbubbles can be configured to carry an active agent (e.g., small molecule) and be acoustically activated. For example, the molecule-carrying microbubbles can carry or be coated with medicinal molecules and/or a contrast agent and/or a biomarker and/or a liposome. Medicinal molecules and/or contrast agents can also be separately positioned in proximity to the targeted region. For example, the active agent can include a monoclonal antibody, a neuronal growth factor, a chemotherapeutic agent, or a combination thereof. In some embodiments, the FUS induced microbubble cavitation can open the target tissue without damaging the target tissue.

In certain embodiments, the disclosed system can include a navigation guidance device that can be configured to locate and/or monitor the target tissue. The navigation guidance device can include a cavitation detector 103 and an arm 104. In non-limiting embodiments, the navigation guidance device can be an image-based navigator device.

In certain embodiments, the cavitation detector 103 can be configured to detect the FUS-induced cavitation in real-time. In non-limiting embodiments, the cavitation detector can be a passive cavitation detector (PCD) co-aligned with the single element transducer. The PCD can have certain imaging parameters that can allow the detection of cavitation signals through a bone (e.g., human skull). For example, the imaging parameter can include a center frequency, a diameter, and a focal depth. The center frequency of the PCD can range from about 0.1 megahertz (MHz) to about 10 MHz, from about 0.1 MHz to about 9 MHz, from about 0.1 MHz to about 8 MHz, from about 0.1 MHz to about 7 MHz, from about 0.1 MHz to about 6 MHz, from about 0.1 MHz to about 5 MHz, from about 0.1 MHz to about 4 MHz, from about 0.1 MHz to about 3 MHz, or from about 0.1 MHz to about 2 MHz. In non-limiting embodiments, the center frequency of the PCD can be about 1.5 MHz. The diameter of the PCD can range from about 10 millimeters (mm) to about 60 mm, from about 10 mm to about 50 mm, from about 10 mm to about 40 mm, from about 20 mm to about 40 mm, or from about 30 mm to about 40 mm. In non-limiting embodiments, the diameter of the PCD can be about 32 mm. The focal depth of the PCD can range from about 30 millimeters (mm) to about 200 mm, from about 30 mm to about 150 mm, from about 40 mm to about 150 mm, from about 50 mm to about 150 mm, or from about 100 mm to about 150 mm. In non-limiting embodiments, the focal depth of the PCD can be about 114 mm.

In certain embodiments, the PCD can detect the cavitation signals to determine the types/modes of the cavitation. For example, the PCD can detect harmonic peaks, ultraharmonic peaks, broadband emissions, a cavitation magnitude, a cavitation duration, and a microbubble velocity to identify stable or inertial cavitation. In stable cavitation, the microbubble expands and contracts with the acoustic pressure rarefaction and compression over several cycles, and such action can result in the displacement of the vessel diameter through dilation and contraction. In inertial cavitation, the bubble can expand to several factors greater than its equilibrium radius and subsequently collapse due to the inertia of the surrounding media, thus also inducing a potential alteration of the vascular physiology. The PCD can detect the cavitation signals that can be used for calculating stable harmonic, stable ultraharmonic, and inertial cavitation levels.

In certain embodiments, the navigation guidance device includes an arm 104. In non-limiting embodiments, the single element transducer 101 co-aligned with the cavitation detector 103 can be attached to the arm 104. The arm can be a robotic arm with 4 degrees of freedom. The movement of the robotic arm can be controlled by a controller 105 (e.g., joystick).

In certain embodiments, the image-based navigator device can be configured to image the target tissue and reconstruct a 3D image before and after the application of the FUS. The 3D skin scalp and brain reconstructions can allow the accurate placing of the focal volume in the targeted region. The planned and achieved trajectory can be visualized in real-time.

In certain embodiments, the disclosed system can further include a transducer tracker 106, a position sensor 107, a radiofrequency amplifier 108, a portable chair 109, and a display 110. The transducer and subject trackers can include infrared light-reflecting spheres and be configured to perform real-time monitoring of the transducer's and subject's position in space. The radiofrequency can amplifies an amplification (e.g., 55-dB) of the signal generated by the function generator before application onto the single-element transducer.

In certain embodiments, the disclosed system can include a processor coupled to the single element transducer and/or the navigation guidance device. In non-limiting embodiments, the processor can be coupled to the probes directly (e.g., wire connection or installation into the probes) or indirectly (e.g., wireless connection). The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components described above. For example, and as embodied herein, the processor can be configured to control imaging and ultrasound stimulation. Additionally, or alternatively, the processor can be configured to control the output of the function generator and/or the transducer to provide the FUS to the subject.

In certain embodiments, the processor can be configured to analyze the detected cavitation signals and determine a mode of the cavitation. The processor can analyze cavitation signals that are measured by the cavitation detector. For example, the processor can calculate stable harmonic, stable ultraharmonic, and inertial cavitation levels by analyzing harmonic peaks, ultraharmonic peaks, broadband emissions, a cavitation magnitude, a cavitation duration, and microbubble velocity signals detected by the PCD. Cavitation doses can be calculated as the sum of cavitation levels throughout the treatment duration. Stable cavitation doses can quantify the magnitude of stable and recurrent cavitation, while inertial cavitation doses can quantify the magnitude of transient inertial cavitation. The relative weighting of stable vs. inertial cavitation can be a safety determinant for ultrasound treatments.

In certain embodiments, the processor can be configured to perform numerical simulations to determine the ultrasound parameter to open the target tissue. The numerical simulation can be used to simulate the effects of the predetermined parameter of a transducer on ultrasound propagation. For example, the processor can perform numerical simulations of ultrasound propagation through the human skull to test different transducer characteristics. In non-limiting embodiments, the processor can identify the trade-off between the focal depth and aperture size (e.g., the f-number) within the target tissue (e.g., within the skull) through numerical simulations. The processor can also determine the ultrasound parameters (e.g., the center frequency, outer diameter, and radius of curvature) that allows opening the target tissue enlarging the treatment envelope. In non-limiting embodiments, the determined ultrasound parameters through the numerical simulations can be used to target both cortical and subcortical regions of the human brain. For example, the numerical simulations can be performed in Matlab using the k-Wave toolbox, which is based on a pseudospectral k-space method to determine complex acoustic wave fields in heterogeneous media. In some embodiments, the numerical simulations can be performed on a patient-by-patient basis, given the CT or MRI scan of a subject, to derive the approximated attenuation factor at a defined target and trajectory.

In certain embodiments, the target tissue can be any tissues. For example, the target tissue can be a nerve, a brain, a heart, muscle, tendons, ligaments, skin, vessels, or a combination thereof. In non-limiting embodiments, the target tissue can be a cortical and.or a subcortical region of a brain.

In certain embodiments, the disclosed subject matter provides a method for opening target tissue. An example method can include locating the target tissue using a navigation guidance device, administering microbubbles into the target tissue, and applying FUS using a single element transducer. In non-limiting embodiments, the navigation guidance device can include a cavitation detector and an arm. The single element transducer can be co-aligned with the cavitation detector and be attached to the arm. The single element transducer can have a predetermined ultrasound parameter to open the target tissue. The predetermined parameter can be selected from the group consisting of a center frequency, an outer diameter, an inner diameter, a radius of curvature, and a combination thereof. In non-limiting embodiments, the predetermined parameter can be adjusted based on the target tissue or the subject.

In certain embodiments, the method can further include obtaining a cavitation signal using the cavitation detector. In non-limiting embodiments, the cavitation signal can be selected from the group consisting of harmonic peaks, ultraharmonic peaks, a broadband emission, a cavitation magnitude, a cavitation duration, and microbubble velocity signals.

In certain embodiments, the method can further include determining a cavitation mode by calculating a stable cavitation dose (SDCh), a stable ultraharmonic (SDCu), and an inertial cavitation dose (ICD) based on the cavitation signal. For example, the SDCh, SDCu, and ICD can be calculated by a processor to determine the cavitation mode.

In certain embodiments, the method can further include determining the predetermined ultrasound parameter for opening the target tissue by performing numerical simulations. For example, the processor can perform numerical simulations of ultrasound propagation through the human skull to test different transducer characteristics. The determined ultrasound parameters (e.g., the center frequency, outer diameter, and radius of curvature) can allow the opening of the target tissue, enlarging the treatment envelope. In non-limiting embodiments, the determined ultrasound parameters through the numerical simulations can be used to target both cortical and subcortical regions of the human brain.

In certain embodiments, the disclosed technique can provide systems and methods for opening target tissue without the need for in-line MM guidance. The disclosed technique can achieve the opening of the target tissue (e.g., blood-brain barrier) at clinically relevant ultrasound exposures. The proposed FUS system can provide non-invasive FUS-mediated therapies due to its fast application, low cost, and portability.

### EXAMPLES

### Example 1: A Clinical System for Non-Invasive Blood-Brain Barrier Opening Using a Neuronavigation-Guided Single-Element Focused Ultrasound Transducer

Numerical simulations: Numerical simulations of ultrasound propagation through the human skull were performed in two dimensions using the k-Wave acoustics toolbox to evaluate different transducer characteristics. The trade-off between the focal depth and aperture size, that is, the f-number, within the human skull, was tested. The disclosed subject matter can be used to determine the center frequency, outer diameter, and radius of curvature to be able to target both cortical and subcortical regions of the human brain, thus enlarging the treatment envelope. Three different transducer configurations (Table 1), which were determined based on commercially available low-frequency models (transducer 1: Sonic Concepts H-149, transducer 2: Sonic Concepts H-209) and a custom-designed transducer (transducer 3), were tested.

TABLE 1
Transducer parameters used in numerical simulations

| | Center | Outer | Inner | Radius of |
|---|---|---|---|---|
| | frequency | diameter | diameter | curvature |
| Transducer | (MHz) | (mm) | (mm) | (mm) |
| 1 | 0.2 | 110 | 44 | 70 |
| 2 | 0.35 | 60 | 44 | 76 |
| 3 | 0.25 | 110 | 44 | 110 |

H-149 and H-209, commercially available models, were chosen as examples of small and large f-number, respectively (0.64 vs. 1.27). The custom-designed transducer (e.g., outer diameter: 110 mm, radius of curvature: 110 mm, f-number: 1) was optimized after multiple iterations of different designs, with emphasis on the outer diameter (e.g., search space: 60-140 mm) and radius of curvature (e.g., search space: 70-120 mm). To allow for the insertion of a PCD transducer or a receiving ultrasound array, an inner gap 44 mm in diameter was applied in all transducer designs.

A human CT skull DICOM file from the Cancer Imaging Archive was used as input in our simulations. Hounsfield CT units were converted to sound speed and medium density. Sound speed, medium density and attenuation coefficient within the brain were set to be equal to those of water at 37° C. (e.g., 1524 m/s, 1000 kg/m3 and 3.5×10⁻⁴ dB/MHz·cm, respectively). The transducers were positioned close to the skull in an effort to place the focal volume as close to the brain median plane as feasable, while maintaining a reasonable radius of curvature and realistic housing dimensions (Table 1). A number of axial offsets were tested (e.g., range: −30 to +30 mm), to determine the evolution of focal shifts across different depths. In the case of an axial offset of 0 mm, the transducer's nominal focus was positioned at the human brain midline. The simulations were performed to evaluate the effect of different focusing depths on the focal volume distortion. Introducing lateral offsets can produce a large variation in the incidence angle, deviating significantly from the desirable 90° incidence. Therefore, the lateral position of the FUS transducer center was fixed at y=0 mm. Pulses with different lengths (e.g., 1, 5, 25 and 2500 cycles) to investigate the effects of interference and standing waves within the human skull. To calculate the theoretical ultrasound transmission coefficient through the human skull, the simulations were repeated with different pulse lengths in free field by replacing the human skull with water. The simulation grid was equal to 300×300 mm, at 1-mm spatial resolution, while the temporal resolution was 143 ns with a total of 7000 times or exposure time of 1 ms. For the pulse length of 2500 cycles, the simulation consisted of 70,000 times or 10 ms, to enable comparison with the treatment scheme used for in vivo BBB opening. Shear waves were not taken into account in these simulations. Axial (i.e., x) and lateral (i.e., y) axes were defined with respect to the FUS transducer, and had left to right and anterior to posterior directions, respectively.

A single transducer clinical system: As shown in FIG. 1, the single transducer clinical system with a low center frequency (e.g., 0.25 MHz) was developed to reduce the attenuation caused by the human skull and decrease the pressure threshold for cavitation induction. The dimensions and characteristics of the single-element spherical-segment transducer were refined based on numerical simulations. Then, the chosen single-element FUS transducer (e.g., center frequency: 0.25 MHz) was constructed and attached it onto a robotic arm. The robotic arm had 4 degrees of freedom and a maximum midrange loading capacity of 4.4 kg, and was controlled via a joystick. The whole construct was fixed onto a wheeled cart, making the system portable to any location.

The clinical FUS transducer was driven by a function generator (33500B Series, Agilent Technologies, Santa Clara, Calif., USA) through a 55-dB radiofrequency power amplifier (A150, E&I, Rochester, N.Y., USA) using clinically relevant parameters (Table 2).

| Parameter | Value | |
|---|---|---|
| Center Frequency | 0.25 | MHz |
| Derated peak-negative pressure | 0.2 | MPapk-neg |
| Mechanical index | 0.4 | |
| Definity microbubble does | 10 μL/kg (1 × clinical dose) | |
| Pulse length | 10 ms or 2500 cycles | |
| Pulse repetition frequency | 2 | Hz |
| Sonication duration | 2 | min |

A water degassing system was used to fill the transducer cone with degassed water and inflate or deflate the cone according to the sonicated location. Reflective beads were attached to the transducer to enable real-time tracking of its location through an infrared camera acting as a position sensor and neuronavigation guidance. Using the bull's eye view function, the disclosed subject matter achieved improved targeting accuracy with spatial error lower than 2 mm.

Microbubble acoustic emissions were recorded (e.g., sampling frequency: 50 MHz, capture length: 10 ms) with a 1.5-MHz passive cavitation detector (PCD: e.g., diameter: 32 mm, focal depth: 114 mm). PCD provides information on the cavitation magnitude, duration and mode within the focal volume, using either separate transducers or a therapeutic transducer alone. Cavitation signals also provide indirect information about the microbubble velocity through the Doppler effect, which can be captured either with a single-element PCD or using an array of receivers. PCD was used to define the cavitation mode in vitro and in vivo by calculating the stable cavitation dose (SCD) and inertial cavitation dose (ICD). The recorded time-domain signals were transformed into the frequency domain through a fast Fourier transform (e.g., segment size: 524,288 data points), performed in MATLAB. Three spectral areas were filtered to derive the relevant cavitation levels or cavitation dose per pulse as follows: 1) Harmonic peaks,f_{h,n}=nf_{c,}2) Ultraharmonic peaks, f_{u,n}=(n−1/2)f_{c}, 3) Broadband emissions f_{b} with f_{h,n}+10 kHz<f_{b}<f_{u,n}−10 kHz and f_{u,n}+10 kHz<f_{b}<f_{h, n+1}−10 kHz. f_{c} is the center frequency (e.g., 0.25 MHz) and n is the harmonic number (e.g., n=3, 4, 5, . . . 10). Fundamental and second harmonics were excluded frequencies in control experiments.

Stable harmonic (dSCDh), stable ultraharmonic (dSDCu) and inertial cavitation (dICD) levels were then calculated as the mean root-mean-square (RMS) of the maximum absolute Fast Fourier Trransform (FFT) amplitude of the detected signal within each frequency region for each acoustic pulse as follows:

dSCDₕ= [symbol] (1)

dSCDᵤ= [symbol] (2)

dICD= [symbol] (3)

The total cavitation does in vivo was calculated as the sum of all the cavitation levels throughout the FUS treatment:

$${SCD}_{h} = {\sum }_{t = 0}^{T} {dSCD}_{h , t} \qquad ( 4 )$$

$${SCD}_{u} = {\sum }_{t = 0}^{T} {dSCD}_{u , t} \qquad ( 5 )$$

$$ICD = {\sum }_{t = 0}^{T} {dICD}_{t} \qquad ( 6 )$$

The total conication duration was 2 min (T=2 min).

In vitro characterization: Skull-induced aberrations were characterized in a water tank. A capsule hydrophone (e.g., ±3-dB frequency range: 0.25-40 MHz, electrode aperture: 200 μm) was used to measure the emitted pressure profiles in free field and with a human skull fragment in the beam path. The skull fragment was submerged in water and degassed before the experiment using a vacuum pump, to reduce the gas content within the bone. Raster scans around the focal point were performed at a spatial resolution of 0.1 mm laterally and 1 mm axially. The scans had lateral/elevational and axial ranges of 10 and 60 mm, respectively, and were centered at the geometric focus of the FUS transducer (e.g., 110 mm from transducer surface). Shifts along the lateral and elevational dimensions were averaged, assuming an axisymmetric distortion of the beam. Ultrasound pressure transmission coefficient through the human skull was calculated (in %) by dividing the maximum pressure of the focal volume after the skull placement by the maximum pressure of the focal volume in free field, for both simulations and experiments. Transcranial pressure loss was calculated as 100% −transmission coefficient. To determine the ultrasound attenuation through an NHP skull, the human skull fragment was replaced with a NHP skull fragment. The human and NHP skull fragments were positioned right on top of the water cone and at a perpendicular incidence angle, to imitate the clinical scenario. Pressure profiles and transcranial loss were expected to be extremely sensitive to the incidence angle and distance from the transducer surface. Incidence angle (e.g., ˜90°) and transducer surface—skull distance (e.g., 62 mm), which are clinically relevant for treatment of dorsolateral prefrontal cortex, were tested. Pressure profiles and losses were estimated at skull locations of variable thickness (e.g., n=10, thickness range: 3-7.5 mm, measured with a caliper), as attenuation depends on the skull thickness. The pressure values refer to the derated peak-negative pressure.

Cavitation detection through the human skull was also conducted within a water tank. A 0.8-mm silicon elastomer tube was submerged and fixed at a horizontal position within the focal volume of the clinical transducer (120 mm from transducer surface). The tube was filled with either water, which served as a control, or Definity microbubbles (0.2 mL microbubbles/L of solution) flowing at a rate of 1.8 mL/min. Measurements were conducted both in free field and with the human skull fragment in the beam path, positioned 62 mm away from the transducer surface. We tested three derated acoustic pressures, 200, 300 and 400 kPa, corresponding to MIs of 0.4, 0.6 and 0.8, respectively. Cavitation levels were calculated across the experimental conditions (n=10 consecutive pulses per condition) to establish the ability of the PCD transducer to detect cavitation signals through the human skull at each acoustic pressure.

A tissue-implantable type-T thermocoupl was attached to the skull surface to measure the heating profile during clinically relevant FUS exposure (e.g., MI: 0.4-0.8, duty cycle: 2%; Table 2). A positive control sonication at a higher duty cycle (20% at an MI of 0.8) was conducted to compare with the low-duty-cycle BBB opening scheme. Temperature data were recorded at a sampling rate of 100 samples/s. Temperature increase on the skull surface was calculated by subtracting the temperature before FUS exposure from the value measured during FUS exposure (e.g., n=3).

In vivo feasibility: All animal testing were reviewed and approved by the local Institutional Animal Care and Use Committee and were in accordance with the National Institutes of Health guidelines for animal welfare. Two male adult Rhesus macaques (e.g. weight: 8-11 kg, age: 12-20 y) were treated with the clinical FUS transducer, targeting the thalamus (NHP 1) and the dorsolateral prefrontal cortex (NHP 2), to examine the performance of the system at both cortical and subcortical regions. To accommodate the NHP experiment, the patient chair (FIG. 1) was replaced with a surgical table equipped with a stereotactic apparatus for head fixation. NHPs were initially sedated with a mixture of ketamine (e.g., 10 mg/kg) and atropine (e.g., 0.02 mg/kg) through intramuscular injection. Once sedated, the animals were intubated and catheterized via the saphenous vein. Anesthesia was induced and maintained throughout the experiment using inhalable isoflurane mixed with oxygen (e.g., 1%-2%).

The ultrasound parameters used here (Table 2) were identical to those approved by the FDA for use in Alzheimer's patients using our system (derated peak-negative pressure: 0.2 MPa, pulse length: 10 ms, pulse repetition frequency: 2 Hz, total sonication duration: 2 min). The MI was maintained below the FDA-approved limit for ultrasound imaging applications with Definity microbubbles to avoid compromising safety. BBB opening in the NHP model was attempted at a peak-negative pressure of 0.2 MPa or an MI of 0.4. This MI is approximately five times lower than the maximum MI approved by the FDA for imaging applications (i.e., MI of 1.9), twice lower than the BBB opening threshold in humans. Commercially available Definity microbubbles were used at the FDA-approved clinical dose for ultrasound imaging applications (e.g., 10 μL/kg). Definity microbubbles were infused as a bolus via a single injection, on treatment initiation.

Blood-brain barrier opening was assessed approximately 60 min post-sonication with T1-weighted MRI (e.g., 3-D spoiled gradient-echo, TR/TE: 20/1.4 ms, flip angle: 30°, number of excitations [NEX]: 2, spatial resolution: 500×500 μm², slice thickness: 1 mm with no inter-slice gap). T1-weighted scans were acquired before and after intravenous administration of 0.2 mL/kg gadodiamide MM contrast agent, which is normally impermeable to the BBB (e.g., molecular weight: 591.7 Da). BBB opening was quantified by comparing pre- and post-contrast administration T1 scans. Safety outcomes were assessed with axial T2-weighted MRI (e.g., TR/TE: 3000/80 ms, flip angle: 90°, NEX: 3, spatial resolution: 400×400 μm2, slice thickness: 2 mm with no inter-slice gap) and susceptibility-weighted imaging (SWI, e.g., TR/TE: 19/27 ms; flip angle: 15°, NEX: 1, spatial resolution: 400×400 μm²; slice thickness: 1 mm with no inter-slice gap). All scans were performed in a 3-T clinical MRI scanner.

BBB opening quantification: a graphics user interface (GUI) was developed in MATLAB for BBB opening quantification and analysis. To calculate the BBB opening volume, the pre-contrast T1 scan was subtracted from the post-contrast T1 scan. An intensity threshold was set to isolate the BBB opening area in the difference image, and a contour plot was applied to the pixels above the threshold within the selected region of interest. The area of the BBB opening contour was calculated for each coronal MRI slice, and the total BBB opening volume (in mm³) was found by summing the BBB opening areas in all slices.

Statistical analysis: Measurements presented are expressed as the mean±standard deviation. Simulations were performed for n=4 pulse lengths and n=6 transducer axial positions. Cavitation detection was established by comparing control and microbubble-seeded cavitation levels in free field and through the human skull, using a two-sample t-test in MATLAB (n=10 pulses). Statistically significant differences were assumed at p<0.05.

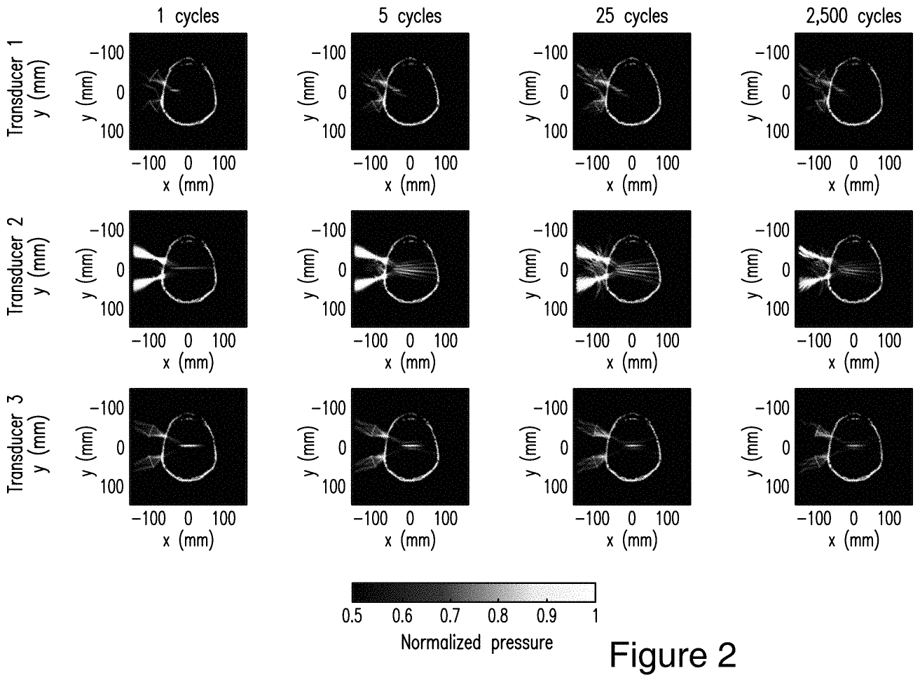

Data—Numerical simulations: Numerical simulations revealed that transducer 3 was able to target the brain median plane while maintaining a tightly focused beam, without multiple sidelobes (FIG. 2). FIG. 2 shows numerical simulations of ultrasound propagation with different single-element transducers (top to bottom: 1, 2, 3) emitting pulses of variable length (left to right: 1, 5, 25, 2500 cycles). Transducer 3 was able to treat deep structures without presenting multiple sidelobes within the human skull. The bar shows normalized focal pressure. Each pressure profile was self-normalized to the maximum acoustic pressure within the skull to illustrate the −3-dB focal volume. Pressure values refer to the maximum instantaneous pressure at each location. Transducer 1 did not have sufficient radius of curvature to produce a long enough focal depth for the human skull, because of its low f-number. Transducer 2 produced multiple sidelobes similar in amplitude to the main lobe because of the large f-number and the low outer-to-inner diameter ratio. Furthermore, the focal volume was subject to greater distortion because of the higher center frequency compared with transducers 1 and 3 (e.g., 0.35 MHz vs. 0.2 MHz and 0.25 MHz). In the case of a single-element transducer, an f-number of 1 (transducer 3) was more suitable for applications in the human brain, compared with lower or larger f-numbers within the tested subset.

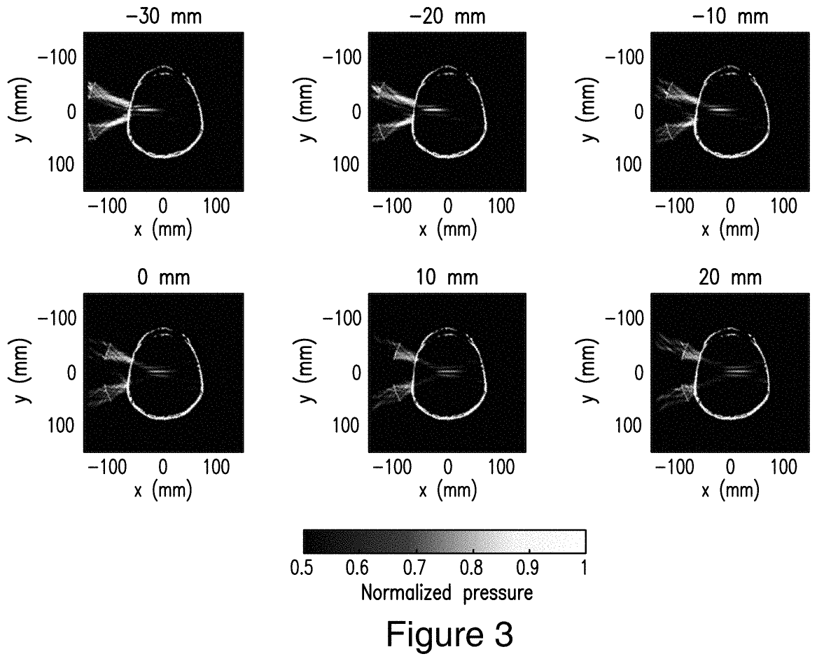

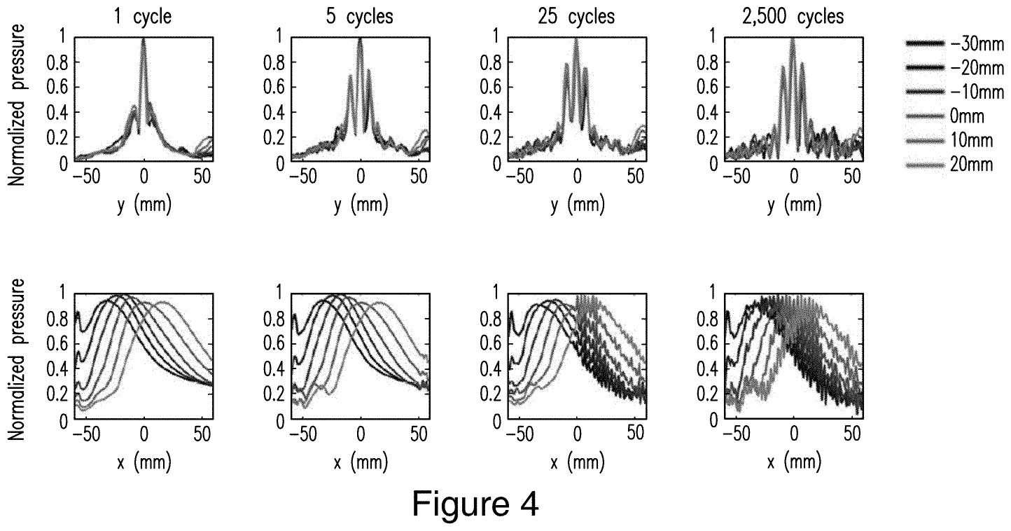

Such a transducer design allows targeting of both superficial cortical areas and deeper subcortical areas (FIG. 3). FIG. 3 shows numerical simulations of ultrasound propagation with the clinical focused ultrasound transducer targeting structures of variable depth within a human skull. In FIG. 3, samples are shown for transducer axial offset of −30 to 20 mm (e.g., offset=0 mm when the focus in free-field coincides with the midline). Center frequenc was about 0.25 MHz, and pulse length was about 2500 cycles. The bar shows normalized focal pressure. Each pressure profile was self-normalized to the maximum acoustic pressure within the skull to illustrate the −3-dB focal volume. Pressure values refer to the maximum instantaneous pressure at each location. By physically moving the FUS transducer toward/away from the skull surface, one can achieve a treatment envelope up to 80 mm in depth. Simulations revealed that the focal dimensions, pressure profile and skull-induced focal shift depend on the transducer axial offset and the pulse length (FIG. 4). FIG. 4 shows lateral (top) and axial (bottom) profiles of the simulated pressure field within a human skull. Lateral sidelobes and interference patterns emerge for pulse lengths larger than one cycle. The spatial length of interference away from the distal skull bone increases linearly with the pulse length. The transducer axial offset was defined as the distance of the free-field focus from the simulation center (e.g., x=0 mm). Intracranial acoustic pressures moderately changed throughout the axial offsets. Highest pressures were observed near the skull center, while there was a decrease of up to 7% toward the proximal and distal skull. The amplitude of lateral sidelobes increased with pulse length, from 49% of the main lobe at 1 cycle to 76% of the main lobe at 2500 cycles. All pressure profiles shown in FIGS. 2 and 3 were normalized to the maximum pressure within the skull and plotted in the range [0.5, 1], to visualize the −3-dB focal volume following transcranial ultrasound propagation.

Pulse lengths longer than 1 cycle produced constructive and destructive interference at the distal part of skull, with nodes and antinodes appearing at a spacing of half-wavelength (e.g., 3 mm). The interference spatial extent was equal to half the spatial length of the acoustic pulse (e.g., 2.5 cycles or 15 mm for a pulse length of 5 cycles or 30 mm). For the clinically relevant pulse length of 2500 cycles, the interference profile reached equilibrium and extended throughout the interior of the human skull. The theoretical limit for standing wave generation at 0.25 MHz and a skull size of 130 mm is 43 cycles.

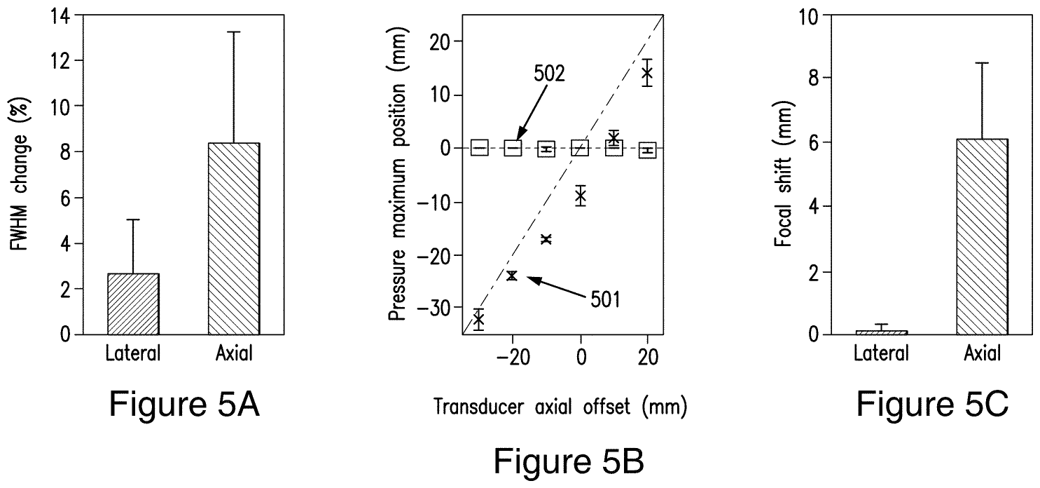

The presence of the human skull led to the distort and spatial shift of the simulated focal volume (FIG. 5). FIG. 5 shows the simulated human skull-induced focal distortion. FIG. 5A shows a full width at half maximum (FHWM) change caused by the presence of the human skull. FWHM changes were first averaged across the pulse lengths for each axial offset (n=4 pulse lengths), and then averaged across all depths (n=6 axial offsets). FIG. 5B shows simulated focal shifts along the axial (crosses: 501) and lateral (boxes: 502) dimensions. Diagonal dotted-dashed line and parallel dotted line denote axial and lateral shifts equal to zero, respectively (n=4 pulse lengths). (c) Average focal shifts across th lateral and axial dimensions (n=6 axial offsets). Data are expressed as the mean±standard deviation. In water medium without the human skull, the axial and lateral full widths at half-maximum (FWHM) were simulated to be 65.5×5.6 mm. The focal width and length were reduced by 2.7±2.4% and by 8.4±4.8% along the lateral and axial dimensions, respectively, because of skull-induced aberrations (n=4 pulse lengths and n=6 transducer positions). The focus was also negatively shifted toward the transducer (FIG. 5B). Axial shifts depended on the transducer position. Interestingly, shifts were smaller for larger offsets. The farther the focus from the brain midline, the smaller the axial shift. On average, the axial and lateral focal shifts were 6.1±2.4 and 0.1±0.2 mm, respectively (FIG. 5C). Pressure attenuation caused by the human skull was simulated to be 36.1±3.4% (e.g., n=10 different CT slices).

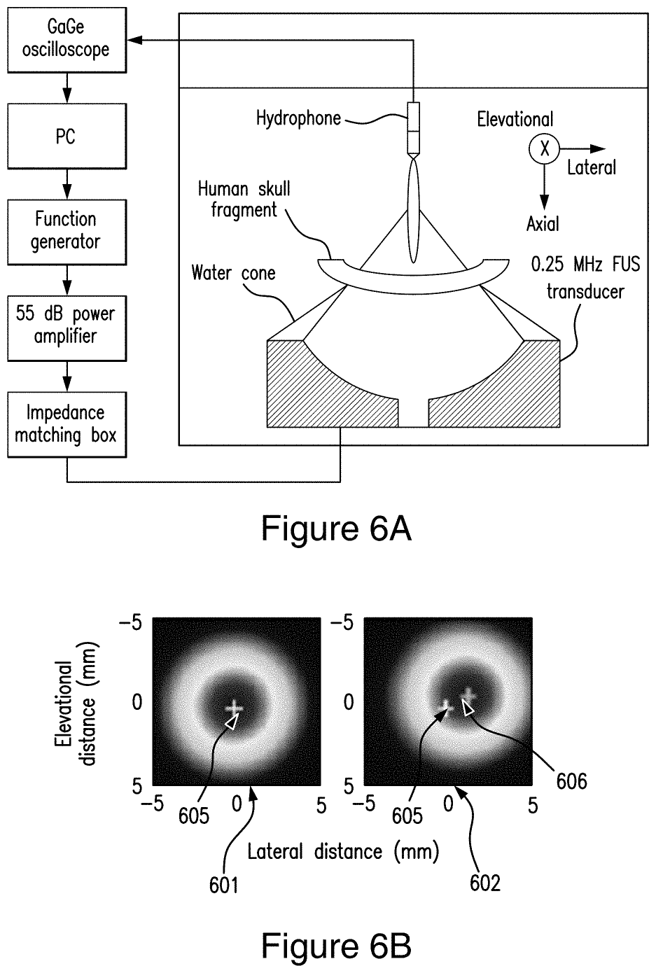

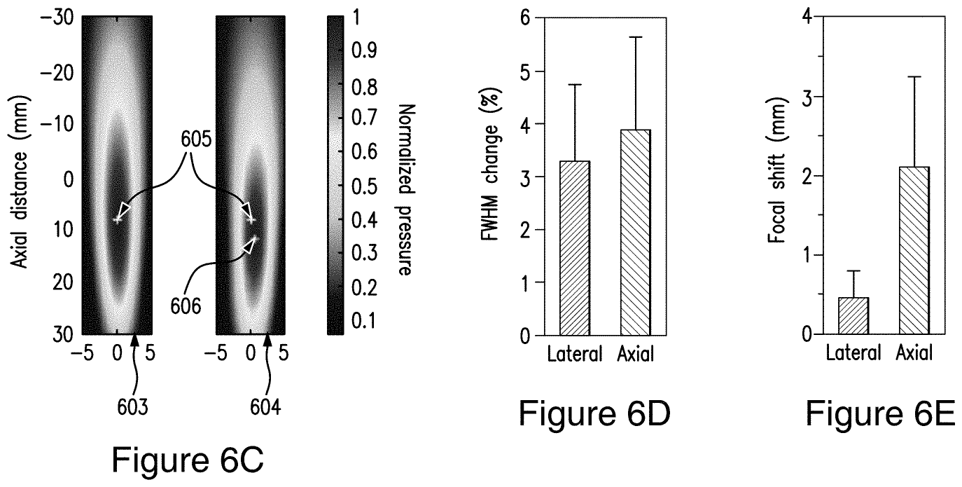

In vitro characterization: To confirm the simulation findings, a detailed estimation of the 2-D beam profiles was performed along the lateral/elevational and lateral/axial dimensions, with and without the presence of a human skull fragment (FIG. 6).

FIG. 6 shows human skull-induced focal distortion. FIG. 6A shows a system for measuring focal distortion using a hydrophone. A raster scan was performed to measure the focal volume in (601 and 603) free field and (602 and 604) with a human skull fragment. Pressure maximum was 10 mm closer to the transducer compared with the geometric focus. First crosses 605 denote the position of the free-field focus. Second crosses 606 denote the position of the focus following transcranial propagation. FIG. 6D shows a full width at half maximum change, and FIG. 6E shows focal shifts along the lateral and axial dimensions. Data are expressed as the mean±standard deviation (n=10 scans with ultrasound propagating through skull segments of different thickness). Using the capsule hydrophone and the 3-D positioning system (FIG. 6A), the pressure profiles were measured along the axial, lateral and elevational dimensions. The free-field focal length and width were 47.6×5.6 mm (FIGS. 6B and 6C: left side). These values were close to the nominal focal dimensions of 49×6 mm provided by the manufacturer. Ultrasound propagation through the human skull was expected to attenuate and shift the acoustic focus. Inserting the skull fragment within the beam path attenuated the pressure amplitude by 44.4±1.3% and distorted the focal region (FIGS. 6B and 6C: right side). The lateral and axial FWHMs decreased by 3.3±1.5% and 3.9±1.8%, respectively (FIG. 6D). Experimental focal shifts along the lateral and axial dimensions were 0.5±0.4 and 2.1±1.1 mm, respectively (FIG. 6E).

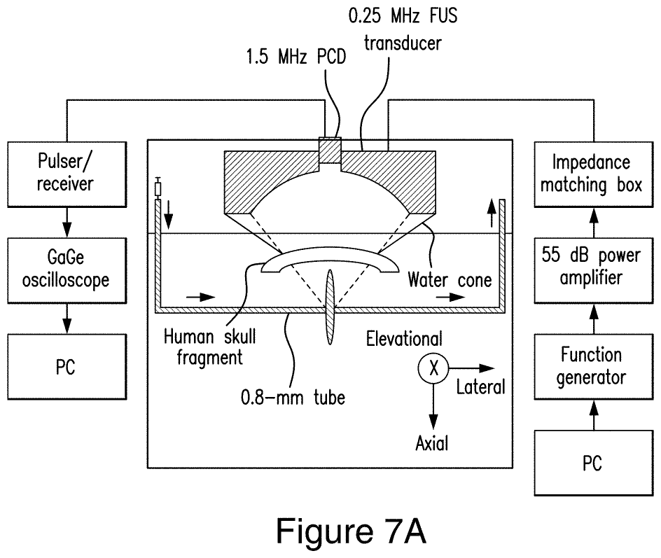

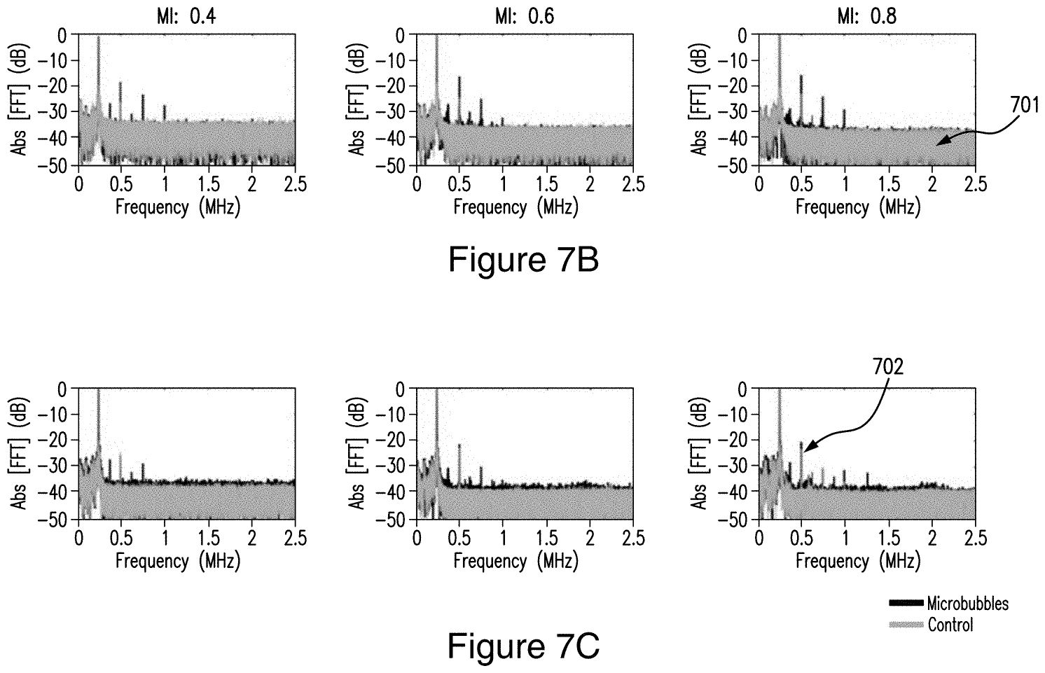

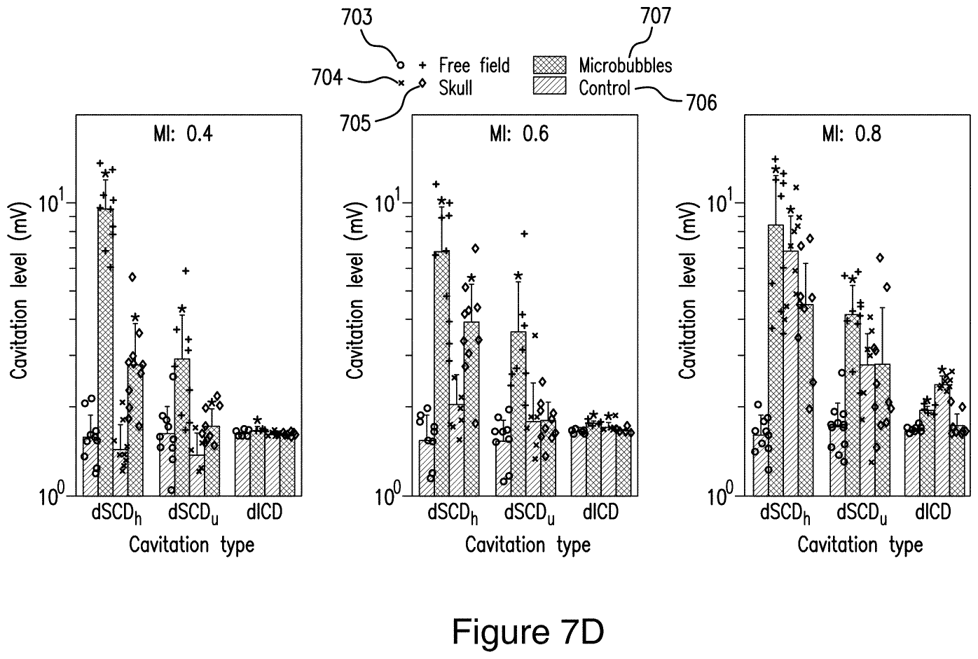

Passive cavitation detection measurements confirmed that the 1.5-MHz PCD transducer can detect cavitation signals through the human skull (FIG. 7).

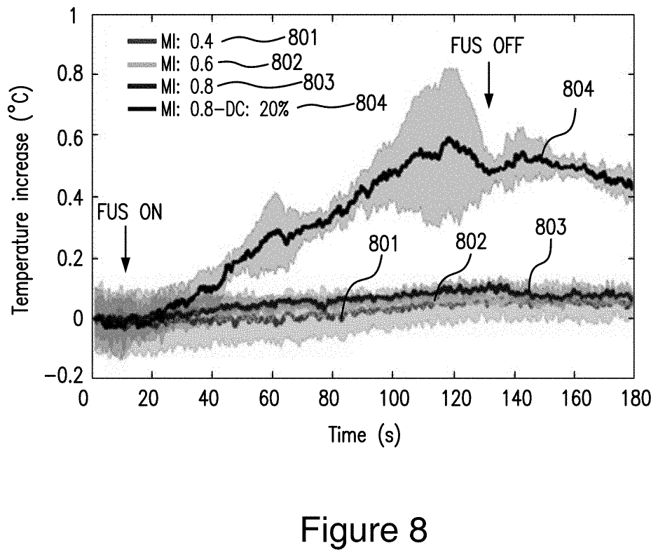

FIG. 7 shows a passive cavitation detection through the human skull. An example system for passive cavitation detection is shown in FIG. 7A. A 0.8-mm tube filled with Definity microbubbles was used as a vessel-mimicking phantom. FIG. 8B shows spectra of control 701 and microbubble 702 acoustic emissions for mechanical indexes (MIs) of 0.4 (left), 0.6 (middle) and 0.8 (right) in free-field. FIG. 7C shows spectra of control and microbubble acoustic emissions through the human skull. FIG. 7D shows cavitation levels in free-field (circles 703) and through the human skull (crosses 704, diamonds 705), for control (light bars 706) and microbubbles (dark bars 707), at MIs of 0.4 (left), 0.6 (middle) and 0.8 (right). Data are expressed as the mean±standard deviation (n=10 pulses).

Using the in vitro system described earlier (FIG. 7A), stationary reflections was observed at the fundamental and the second harmonic for the control experiment, both from the tube and from the human skull (FIG. 7B). When Definity microbubbles were flowing through the vessel phantom, a rise was observed in the higher harmonics (e.g., up to the fifth harmonic or 1.25 MHz) and ultraharmonics (e.g., up to the third ultraharmonic or 0.825 MHz).

Higher acoustic pressures led in general to higher harmonic and ultraharmonic peaks. In FIG. 7d, light-color bars represent control sonications, while dark-color bars represent sonications with Definity microbubbles. The two leftmost bars in each cavitation dose represent free-field sonications, while the two rightmost bars represent sonications through the human skull fragment. Ten distinct therapeutic pulses were emitted for each condition.

Harmonic stable cavitation levels were significantly higher for microbubbles than the control, for MIs of 0.4 and 0.6 both in free-field and through the human skull (FIG. 7D). Ultraharmonic stable cavitation levels with microbubbles were significantly higher than those of the control at MIs of 0.4 and 0.6 only in free-field. There was a significant difference through the human skull at an MI of 0.4, but a non-significant increase at an MI of 0.6. At the highest acoustic pressure, stable harmonic and inertial cavitation levels were significantly higher for the control than for microbubbles. This was likely due to inadequate degassing of the human skull fragment, which resulted in intracranial cavitation nuclei in the control experiment. Inertial cavitation levels rose considerably above the noise level at all MIs in free-field, and also during the control experiments in the presence of skull for MIs of 0.6 and 0.8.

The ultrasound-induced heating was measured during clinically relevant ultrasound exposure. A wire thermocouple was attached below the human skull fragment and within the ultrasound beam path. To simulate the clinical scenario, 2-min sonications were performed using the parameters intended for the clinic (Table 2). The maximum temperature increase was between 0.11±0.05° C. and 0.16±0.03° C. (n=3) during sonication at MIs of 0.4-0.8 (FIG. 8). FIG. 8 shows skull heating using the clinical focused ultrasound transducer at mechanical indexes (MIs) of 0.4 (801), 0.6 (802) and 0.8 (803) and clinically relevant ultrasound parameters (center frequency: 0.25 MHz, pulse length: 2500 cycles or 10 ms, pulse repetition frequency: 2 Hz, duty cycle: 2%, total duration: 2 min). A higher duty cycle (i.e., DC: 20%) was used as a positive control for heating 804. Data are expressed as the mean±standard deviation (n=3). This negligible heating was expected, given the low duty cycle of ultrasonic pulse sequences used in BBB opening (e.g., 2%). A control sonication at 10×higher duty cycle (e.g., 20%) and an MI of 0.8 did increase the temperature by 0.59±0.23° C.

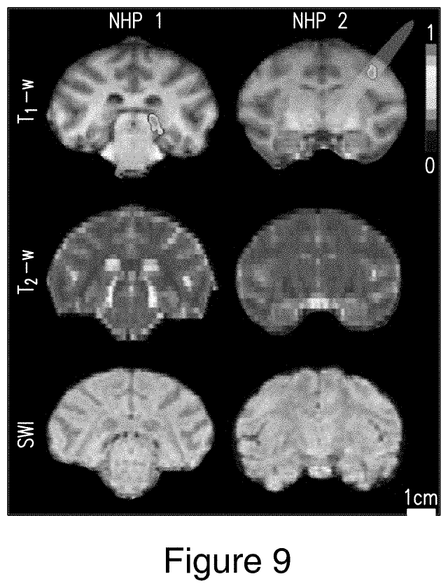

In vivo feasibility: the disclosed clinical system was used to perform non-invasive and targeted BBB opening for an NHP model at a peak-negative pressure of 200 kPa or an MI of 0.4, using the clinically recommended Definity dose (e.g., 10 μL/kg). Two NHPs were treated targeting the thalamus (NHP 1) and the dorsolateral prefrontal cortex (NHP 2). The two targets were selected as examples of deep and superficial structures, respectively. Despite the low pressure and microbubble dose, BBB opening were observed in both targeted structures (FIG. 9). BBB opening was more pronounced in the gray matter rather than in the white matter tracts. The total BBB opening volume was 153 mm3 for NHP 1 and 164 mm3 for NHP 2. Safety was evaluated with T2-weighted MRI and SWI (FIG. 9). Coronal T1-weighted, T2-weighted and susceptibility-weighted imaging (SWI) for NHPs 1 (left) and 2 (right) were shown in FIG. 9. T1-Weighted magnetic resonance imaging-confirmed blood—brain barrier opening in the thalamus (NHP 1) and dorsolateral prefrontal cortex (NHP 2), using the clinical focused ultrasound (FUS) transducer with clinically relevant parameters (MI: 0.4) and microbubble dose (10 μL/kg). T2-Weighted imaging and SWI revealed that there is no acute hemorrhage or edema after the FUS treatment. There was neither a hyper-intense region in T2 scans nor a hypo-intense region in SWI an hour post-sonication, indicating lack of hemorrhage or edema in the sonicated region.

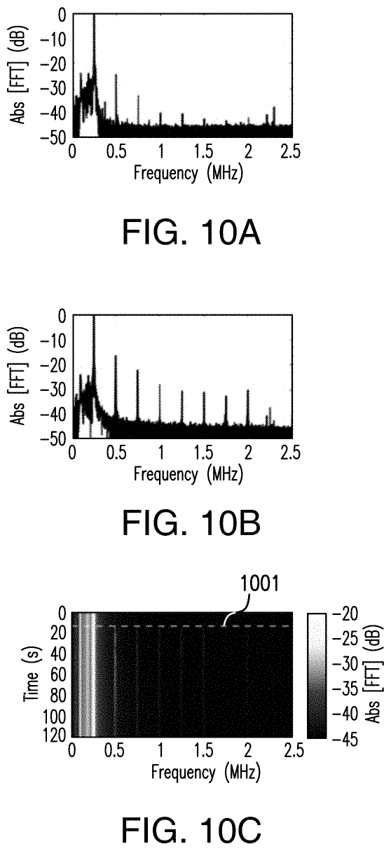

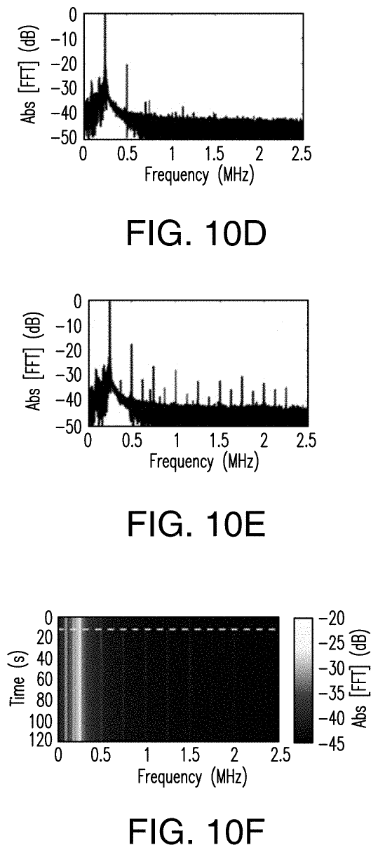

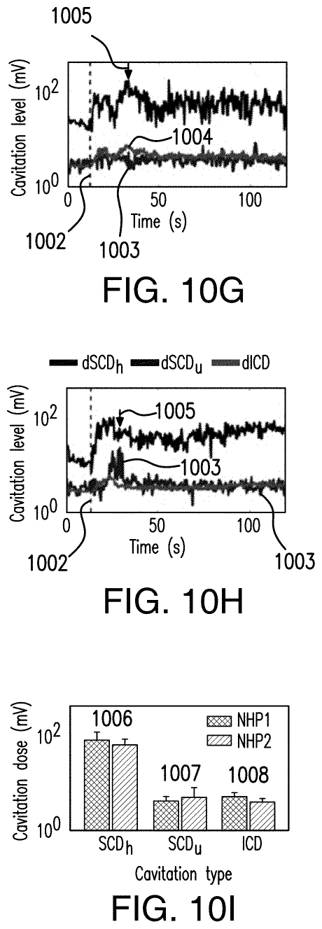

Safety outcomes were corroborated by the captured PCD data which confirmed in real time the absence of violent cavitation events within the focal volume (FIG. 10). In vivo passive cavitation detection measurements confirmed that stable cavitation dominated throughout ultrasound treatment at clinically relevant conditions. Spectral amplitude (FIGS. 10A and 10D) before and (FIGS. 10B and 10E) after microbubble injection, for non-human primate (NHP) 1 and NHP 2 are shown. Spectrogram of the entire treatment session for NHP 1 (FIG. 10C) and NHP 2 (FIG. 10F). Higher harmonic emissions were detected, with no substantial increase in the broadband floor after microbubble entrance into the focal volume (dashed line: 1001). FIGS. 10G and 10H shows stable harmonic cavitation levels rose right after microbubble administration (dashed line: 1002) and remained relatively constant throughout the sonication, for both NHP 1 and NHP 2. Stable ultraharmonic 1003 and inertial cavitation levels 1004 had a moderate increase, indicating absence of violent cavitation events at an MI of 0.4. Arrows 1005 indicate the time points shown in FIGS. 10B and 10E. FIG. 10I shows average stable harmonic (1006), stable ultraharmonic (1007) and inertial (1008) cavitation dose during focused ultrasound treatment for NHP 1 (filled bars) and NHP 2 (patterned bars), following microbubble administration (t>15 s). Data are expressed as the mean±standard deviation (n=210 pulses). Before microbubble administration, the spectral content of the received signals included the fundamental frequency (e.g., 0.25 MHz) and the first two or three harmonics (FIGS. 10A and 10D). Following microbubble bolus injection, there was an increase in higher harmonics and, for NHP 2, ultraharmonics (FIGS. 10B and 10E). However, there was no considerable increase in the broadband signal floor following microbubble administration, as illustrated in the spectrograms of both FUS treatments (FIGS. 10C and 10F). These qualitative traits were quantified with SCD and ICD (FIGS. 10G-10I). SCDh increased by 5.44±1.16-fold on microbubble infusion (t>15 s), while SCDu and ICD increased by 1.46±0.01- and 1.48±0.21-fold, respectively. Microbubbles underwent stable and recurrent oscillations, with stable cavitation dominating over transient and inertial cavitation throughout treatment. On average, the total cavitation dose was 1.37±0.17×104 mV.

A clinical system using a single-element transducer and neuronavigation guidance for BBB opening offers distinct advantages compared with alternative approaches. First, BBB opening can be achieved in a non-invasive manner, which can be advantageous especially for long-term repeated treatments required in AD or PD. Second, such a system can provide access to both shallow (i.e., cortical) and deep (i.e., subcortical) brain regions (FIGS. 3-5), although at the expense of a large axial-to-lateral focal size ratio and variable focal distortion in different depths (FIG. 5). Also, there is no need for an MRI system during treatmentBBB opening which can be a costly and formidable hurdle for widespread use of FUS-mediated treatments, especially given that temperature elevation is not incurred. Neuronavigation systems are available for neurosurgical operations, so the only additional cost for hospitals is the single-element transducer, the driving electronics and the robotic arm. The targeting and sonication are efficient and simple (<30 min) as opposed to MR-guided FUS treatment (e.g., 3-4 h). Moreover, the NgFUS is portable so treatment can take place at any location without the need of an MRI unit. Low-frequency and low-duty-cycle treatment leads to limited skull-induced aberrations (FIGS. 5 and 6) and FUS-induced skull heating (FIG. 8), respectively.

Lower frequencies favor cavitation-mediated bio-effects at low acoustic pressures. The BBB can be opened in an NHP model at an MI of 0.4 (FIG. 9), which is twice lower than the minimum MI required using the unfocused implanted 1.05-MHz transducer in humans. Low-pressure treatments not only ensure safety (FIG. 9), but also facilitate regulatory approval because they are compatible with routinely used ultrasound imaging protocols. Such acoustic pressure instigates cavitation activity that is detectable in real time with the co-aligned PCD transducer (FIG. 7), with stable cavitation emissions dominating the spectra during FUS treatment in an NHP model (FIG. 10). Clinically relevant parameters (Table 2) are thus not expected to lead to violent inertial cavitation, which was detected in higher-MI sonication (FIG. 7).

Successful BBB opening was performed using 10-ms-long pulses. In certain embodiments, the disclosed subject matter can use shorter pulses on the order of microseconds (<50 cycles) to avoid standing wave formation. Short pulses can allow for improved passive mapping of cavitation signals through the synchronization of the therapeutic and imaging processes (e.g., using absolute time-of-flight information). PAM in either the time or frequency domain can be achieved by replacing the single-element PCD transducer with a multi-element linear array operating in receive mode. Using a PAM array, one can account for skull-induced aberrations in receive and localize acoustic cavitation activity in a more precise manner.

Using the disclosed system, numerical simulations were performed in 2-D space, assuming axisymmetric beam profiles along the axial dimension. The human skull is asymmetric and highly inhomogeneous in 3-D space, therefore the simulated profiles are a first-order approximation. The single-element transducer was simulated in k-Wave as a collection of 1-mm point sources firing simultaneously. To test effects of focusing the therapeutic beam at different depths (FIGS. 3-5), incidence angles (e.g., approximately 90°) were set for both simulations. In some embodiments, the lateral position of the FUS transducer remained constant in the numerical simulations. There was a discrepancy between the simulated and experimental pressure losses following transcranial propagation (36% vs. 44.4%), which can be reduced by using 3-D simulations, finer grids and time, and identical skull shapes/dimensions. The disclosed subject matter can be used for 3-D simulations for each patient, using a grid with an isotropic resolution of 0.5 mm, a specific beam trajectory, and a well-defined target within the prefrontal cortex.

On average, axial shifts were of similar magnitude to those predicted in simulations than in the experiments (FIGS. 5 and 6). Averaging in the simulations was conducted over different pulse lengths and focusing depths (FIG. 5), whereas experimental measurements (FIG. 6) were conducted with a single pulse length (e.g., 25 cycles) and fixed transducer—skull distance (e.g., 62 mm). The axial shift in the simulation, which resembled the experimental skull—transducer distance (e.g., the axial offset of −30 mm) was 2.25±1.92 mm (FIG. 5), similar to the experimentally derived shift of 2.1±1.1 mm (FIG. 6). The in vitro cavitation detection experiment was conducted using a single 0.8-mm vessel-mimicking tube, which does not capture the complexity and variability of the in vivo vasculature. Although all simulations and bench-top experiments focused on the human skull, the initial in vivo feasibility testing of the NgFUS system was conducted using two NHPs.

The disclosed subject matter provides a clinical system for BBB opening based on a single-element transducer with neuronavigation guidance and real-time cavitation monitoring. Using this system, one can achieve non-invasive and targeted BBB opening with limited focal distortion and induced skull heating. Lateral and axial shifts were experimentally measured to be 0.5±0.4 and 2.1±1.1 mm and were simulated as 0.1±0.2 and 6.1±2.4 mm. The focal volume decreased by 3.3±1.4% and 3.9±1.8% along the lateral and axial dimensions, respectively, following transmission through a human skull fragment. The maximum temperature increase on the skull surface was 0.16±0.03° C. Using this clinical system, a 153±5.5 mm³ BBB opening was performed in an NHP model with clinically relevant parameters and without any detectable damage.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein.

Such equivalents are intended to be encompassed by the following claims.

What is claimed is:

1. A system for opening a target tissue of a subject, comprising:
a navigation guidance device configured to locate and/or monitor the target tissue, comprising
a cavitation detector, and
an arm;
a single element transducer, coupled to the arm, for stimulating the target tissue with focused ultrasound (FUS), wherein the single element transducer induces the FUS with a predetermined parameter to open the target tissue; and
a processor configured to determine a cavitation mode.

2. The system of claim 1, wherein the arm is configured to have 4 degrees of freedom and be controlled by a controller.

3. The system of claim 1, wherein the single element transducer is connected to a function generator.

4. The system of claim 1, further comprises microbubbles configured to react to the FUS.

5. The system of claim 4, wherein the microbubbles are configured to react a predetermined pulse of the FUS and induce cavitation for opening the target tissue.

6. The system of claim 4, wherein a size of the microbubbles ranges from about 1 micon to about 10 microns.

7. The system of claim 4, wherein the microbubbles are configured to carry or be coated with an active agent.

8. The system of claim 1, wherein the cavitation detector is configured to detect the microbubble cavitation.

9. The system of claim 6, wherein the cavitation detector is configured to capture a cavitation signal, wherein the cavitation signal is selected from the group consisting of a cavitation magnitude, a cavitation duration, and a microbubble velocity.

10. The system of claim 1, wherein the processor is configured to determine a stable cavitation dose (SCD) and an inertial cavitation dose (ICD) based on the cavitation signal.

11. The system of claim 1, wherein the navigation guidance device is an image-based navigator device.

12. The system of claim 1, wherein the predetermined parameter to open the target tissue is selected from the group consisting of a center frequency, an outer diameter, an inner diameter, a radius of curvature, and a combination thereof, and wherein the processor is configured to determine a value of the predetermined parameter through numerical simulations.

13. The system of claim 12, wherein the center frequency ranges from about 0.2 MHz to about 0.35 MHZ.

14. The system of claim 12, wherein the outer diameter ranges from about 60 mm to about 110 mm, wherein the radius of curvature ranges from about 70 mm to about 110 mm, and wherein the inner diameter is about 44 mm.

15. The system of claim 1, wherein the target tissue comprises a cortical brain structure, a subcortical brain structure, or a combination thereof.

16. A method for opening a target tissue of a subject, comprising:
locating the target tissue using a navigation guidance device, wherein the navigation guidance device comprises a cavitation detector and an arm;
administering microbubbles into the target tissue; and
applying FUS using a single element transducer, wherein the single element transducer induces the FUS with a predetermined parameter to open the target tissue, the predetermined parameter is selected from the group consisting of a center frequency, an outer diameter, an inner diameter, a radius of curvature, and a combination thereof.

17. The method of claim 16, further comprising:
obtaining a cavitation signal using the cavitation detector, wherein the cavitation signal is selected from the group consisting of a cavitation magnitude, a cavitation duration, and a microbubble velocity.

18. The method of claim 17, further comprising:
determining a cavitation mode by calculating a stable cavitation dose (SCD) and an inertial cavitation dose (ICD) based on the cavitation signal.

19. The method of claim 16, further comprising:
determining the predetermined parameter by performing numerical simulations.
